(19) United States
(12) Patent Application Publication
(10) Pub. No.: US 2004/0254567 A1
(43) Pub. Date: Dec. 16, 2004

(54) Surgical method for ablating tissue

(76) Inventors: Frank G. Holz, Bonn (DE); Florian Schutt, Heidelberg (DE); Almut Bindewald, Bonn (DE); Holger Specht, Aalen Unterrombach (DE)

Correspondence Address: Brian P. Hopkins, Esq., Mintz, Levin, Cohn, Ferris, Glovsky & Popeo, P.C., The Chrysler Center, 666 Third Avenue, 24th Floor, New York, NY, 10017, US

(21) Appl. No.: 10/778,642

(22) Filed: Feb. 12, 2004

Related U.S. Application Data
(60) Provisional application No. 60/446,773, filed on Feb. 12, 2003.

(51) Int. Cl. A61B 18/18

(52) U.S. Cl. 606/4; 606/16; 606/12; 607/89; 607/101; 601/2

(57) ABSTRACT

The present invention relates to surgical methods which may include excising a sheet of tissue from surrounding tissue, treating the isolated sheet with focused energy thereby ablating at least one layer of said sheet (or a part thereof) and inserting the treated sheet to the location from which the sheet of tissue was excised or to another location. The invention also includes devices for conducting such methods. The method is particularly useful for the treatment of age-related macular degeneration.

### CLAIM TO PRIORITY

This application claims benefit under 35 U.S.C. § 119(e) of U.S. Provisional Application No. 60/446,773, filed Feb. 12, 2003, the entire contents of which are incorporated herein by reference.

### FIELD OF THE INVENTION

This invention relates to a surgical method that is particularly useful for the removal of choroidal neovascularizations which are associated with macular degeneration.

### BACKGROUND OF THE INVENTION

Age-related macular degeneration (AMD) is the leading cause of legal blindness of people over in western nations 50 years of age. The most frequent cause for severe visual loss associated with AMD is the growth of neovascular membranes from a choroid into the subretinal space [Freund, K. B., Yannuzzi, L. A., J. A. Sorenson: Age-related macular degeneration and choroidal neovascularization, Am. J. Ophthalmol. 115 (1993) 786-791]. This usually results in irreversible degeneration of the overlying retina.

Various therapeutic modalities have been brought forward in patients with AMD including thermal laser photocoagulation [Macular Photocoagulation Study Group: Argon laser photocoagulation for neovascular maculopathy: three-year results from randomized clinical trials, Arch. Ophthalmol. 104 (1986) 694-701], surgical removal of the neovascular net with or without transplantation of retinal pigment epithelial or iris pigment epithelial cells [Algvere, P. V., Beglin, L., Gouras, P. Y. Sheng: Transplantation of fetal retinal pigment epithelium in age-related macular degeneration with subfoveal neovascularization, Graefes Arch. Clin. Exp. Ophthalmol. 232 (1994) 707-716], macular translocation/rotation [Eckardt C., Eckardt U., H. G. Conrad: Macular rotation with and without counter-rotation of the globe in patients with age-related macular degeneration, Graefes Arch. Clin. Exp. Ophthalmol. 237 (1999) 313-325], photodynamic therapy [Woodburn, K. W., Engelman, C. J., M. S. Blumenkranz: Photodynamic therapy for choroidal neovascularization: a review, Retina 22 (2002) 527-528] or pharmacological therapy using antiangiogenic drugs [Challa, J. K., Gillies, M. C., P. L. Penfold: Exsudative degeneration and intravitreal triamcinolone, 18 month follow up, Aust. N. Z. J. Ophthalmol, 26 (1998) 277-281], and radiotherapy [RAD Study Group: A randomized, prospective, double-blind clinical trial on radiation therapy for subfoveal choroidal neovascularization secondary to age-related macular degeneration, Ophthalmology 106 (1999) 2239-2247]. However, the prognosis for improved vision is still dismal for most of the patients.

As age-related macular degeneration represents a major health burden with increasing prevalence due to demographic changes, development of novel and effective modes of therapy appears mandatory.

Microsurgical excision of neovascular membranes via a small retinotomy temporal superior to the macula during pars plana vitrectomy is technically, relatively easily accomplished but is usually associated with inadvertent removal of the underlying RPE due to tight adherence of the RPE to the membrane. As functional RPE cells are a prerequisite for normal photoreceptor function, this usually leads to a blind spot in the central retina and, therefore, to loss of some abilities (e.g. reading ability) Only an intact, functioning monolayer of RPE cells beneath the neurosensory retina could maintain regular metabolism and retinal function [Bird, A. C.: Age-related macular disease, Br. J. Ophthalmol. 80 (1996) 1-2; Holz, F. G., D. Pauleikhoff, R. F. Spaide, A. C. Bird: Age-related Macular Degeneration, Springer-Verlag (2003)].

Several attempts have been undertaken to replace removed RPE during surgical excision of subfoveal choroidal neovascularizations. Allogenic RPE was apparently rejected and resulted in accumulation of extracellular fluid with chronic macular edema and loss of visual function [Rezai, K. A., Semnani, R. T., Farrokh-Siar, L., Hamann, K. J., Patel, S. C., Ernest, J. T., G. A. van Seventer: Human fetal retinal pigment epithelial cells induce apoptosis in allogenic T-cells in a Fas ligand and PGE2 independent pathway, Curr. Eye Res. 18, (1999) 430-439]. Autologous cultured iris pigment epithelial cells injected subretinally as cell suspension were not associated with functional recovery [Thumann, G., Aisenbrey, S., Schraermeyer, U., Lafaut, B., Esser, P., Walter, P., K. U. Bartz-Schmidt: Transplantation of autologous iris pigment epithelium after removal of choroidal neovascular membranes, Arch. Ophthalmol. 118 (2000) 1350-1355]. Another approach has been the excision from extramacular RPE/choroid complexes that were transferred at the subretinal site where the neovascular tissue had been removed before [Stanga, P. E., Kychenthal, A., Fitzke, F. W., Halfyard, A. S., Chan, R., Bird, A. C., G. W. Aylward: Retinal pigment epithelium translocation and central visual function in age-related macular degeneration: preliminary results, Int. Ophthalmol. 23 (2001) 297-307; Stanga, P. E., Kychenthal, A., Fitzke, F. W., Halfyard, A. S., Chan, R., Bird, A. C., G. W. Aylward: Retinal pigment epithelium translocation after choroidal neovascular membrane removal in age-related macular degeneration, Int. Ophthalmology 109 (2002), 1492-1498; van Meurs, J., Averst, E., Stalman, P., Kuijpers, R., Hofland, L., Baarsma, S., M. van Hagen: The translocation of autologous retinal pigment epithelial cells in patients with subfoveal choroidal neovascularization, Club Gonin Meeting 2002; J. C. van Meurs and P. R. van den Biesen: Autologous Retinal Pigment Epithelium and Choroid Translocation in Patients With Exsudative Age-related Macular Degeneration: Short-term Follow-up, Am J Ophthalmol 136 (2003) 688-695]. Although retinal function was detected over these grafts, visual outcome was overall unsatisfactory.

### SUMMARY OF THE INVENTION

It is an object of the present invention to provide an effective method for treating tissue, particularly damaged or degenerated tissue, including, for example, choroidal neovascularizations.

It is another object of the present invention to provide an effective method for the treatment of macular degeneration.

These and other objects may be solved by a surgical methods and associated devices according to some of the embodiments of the invention, a summary of which is provided below.

Accordingly, in one embodiment of the invention, a surgical method is presented which may include excising a first sheet of tissue from surrounding tissue at a particular location, treating the excised sheet of tissue with focused energy to ablate at least one layer of the sheet, or a part thereof and inserting the treated sheet back to the location or to another location.

In another embodiment of the invention, a surgical method is provided which may include excising damaged and/or degenerative tissue from a particular location, isolating from the damaged and/or degenerative tissue a sheet of non-damaged and/or non-degenerated tissue comprising at least two layers of tissue, treating the isolated sheet with focused energy to ablate at least one layer of tissue or a part thereof and inserting the treated sheet of tissue back to the location.

In another embodiment of the invention a surgical method is provided which may include excising tissue comprising at least one choroidal neovascularization from a particular location, isolating a sheet of tissue comprising choroidal tissue from the excised tissue, treating the isolated sheet of tissue with focused energy to ablate all or a part of the choroidal tissue and inserting the treated sheet of tissue back to the location.

In yet another embodiment, a surgical method is provided which may include excising at least one choroidal neovascularization from a neurosensory retina at a particular location, isolating a sheet of tissue comprising choroidal tissue, Bruch's membrane and retinal pigment epithelial cells, treating the isolated sheet with focused energy to ablate all or a part of the choroidal tissue and inserting the treated sheet which includes the retinal pigment epithelial cells back to the location.

In still yet another embodiment of the invention, a method for the treatment of macular degeneration is provided which may include excising at least one choroidal neovascularization from a particular location, isolating a sheet of tissue from the choroidal neovascularization comprising choroidal tissue, Bruch's membrane and retinal pigment epithelial cells from the peripheral retina, treating the isolated sheet of tissue with an Excimer laser thereby ablating all or a part of the choroidal tissue and inserting the treated sheet of tissue including the retinal pigment epithelial cells and the Bruch's membrane back to the location.

The above embodiments, other objects, advantages and feature of the invention will become even more evident with reference to the attached figures and detailed description set out below.

### BRIEF DESCRIPTION OF THE DRAWINGS

FIG. 1 is a graphical illustration of a preferred method of the present invention for the treatment of macular degeneration.

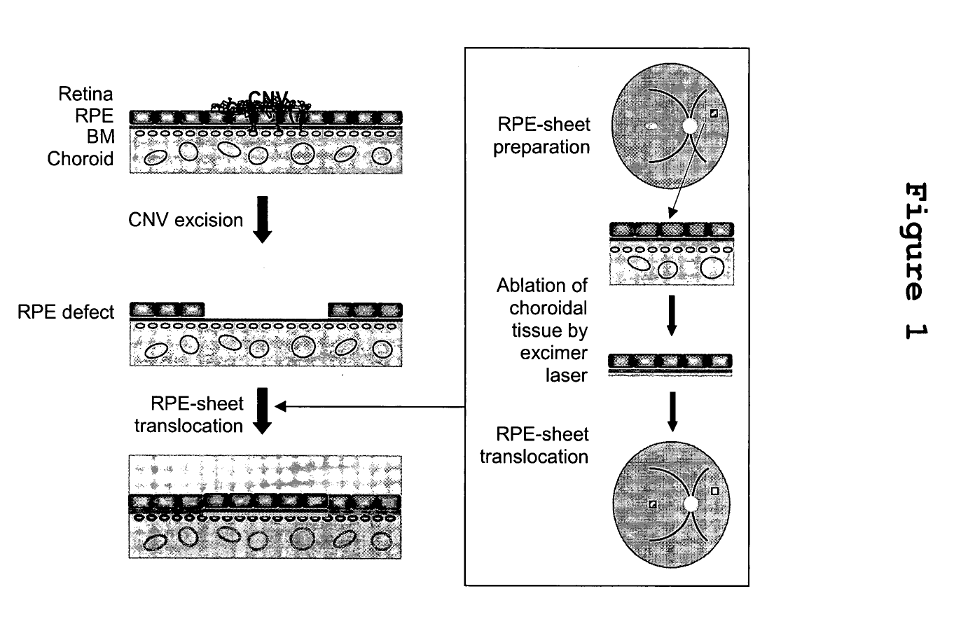

FIG. 2 is a photograph showing the front and side view of a laser tip with a modified aperture (e.g., 300 μm) which may be used to perform methods according to some embodiments of the present invention.

### DETAILED DESCRIPTION OF SOME OF THE EMBODIMENTS

According to the present invention, a sheet of tissue is isolated from surrounding tissue and then treated with, for example, laser light to ablate at least one layer of the sheet, or a part thereof. Other forms of focused energy may be used in addition to or in place of the laser light. Such focused energy may include ultrasound and high-frequency electrical energy, for example.

Although the isolated tissue may be damaged or degenerated, it may also be a non-damaged or non-degenerated tissue isolated from another region of the patient's body (e.g. patient's eye). The term “non-damaged” and “non-degenerated” mean that substantially no physiological dysfunction of the tissue is apparent.

The tissue may be composed of two or more layers. At least two layers of such a laminate may have different ablation characteristics at the wavelength used for ablation. The term “different ablation characteristics” may be defined as layers showing different ablation thresholds at the laser wavelength. The difference in the ablation characteristics are generally inherent to the tissue. However, in those cases where the inherent differences are not sufficient for a proper ablation, light absorbers (e.g. UV absorbing substances) may be introduced specifically to the layer to be ablated. The ablation characteristics may be determined by means of a empirical determination of the necessary ablation threshold for each layer. Investigations have shown that the choroidal capillaries serve as boundary layer with restraining function to the ablation with 308 nm laser light.

The surface area of one side of the isolated sheet is preferably in the range of 1 to 30 mm², and more preferably 5 to 20 mm². In the case that the isolated sheet is to be transplanted after the laser treatment, the size of the isolated sheet is preferably selected such that it corresponds to the size of the excised damaged or degenerated tissue. The tissue preferably includes choroidal tissue, Bruch's membrane and RPE cells and is preferably derived from the peripheral retina. The isolation can be carried out by surgical excision. Initially, this site is demarcated with laser to avoid bleeding from retinal or choroidal vessels. Excision is then performed with retinal scissors. This step could potentially also be carried out with an Excimer laser. The autologous patch to be translocated may then be held with forceps.

The isolated sheet is subsequently treated with a laser light in a manner that allows the ablation of that tissue layer having the lower ablation threshold at the laser light wavelength. This can be achieved by selecting a laser that operates at a wavelength at which the tissue layer to be ablated has a lower ablation threshold than the adjacent tissue layers. The laser light wavelength can be in the UV/VIS range (i.e. from 180 to 800 nm).

For the ablation of choroidal tissue, the laser is preferably one emitting light at a wavelength in the range of 180 to 400 mn. For example, a laser operating at 193 nm (e.g. a ArF Excimer laser) may be used. However, longer wavelengths in the range of 280 to 400 nm are preferred, a laser operating at 308 nm (e.g. a XeCl Excimer laser) being particularly preferred. Alternatively, laser diodes, femtosecond lasers, Er:YAG lasers or Nd:YAG lasers, which may be frequency-multiplied, may be used. Longer wavelengths allow the ablation to be carried out in the eye, i.e. intraocularly.

The 308 UV Excimer laser is known to be suitable for the ablation of human tissue including skin cartilage [Kaufmann, R., R. Hibst: Pulsed Er:YAG- and 308 nm UV-Excimer laser: an in vitro and in vivo study of skin ablative effects, Lasers Surg. Med., 9 (1989) 132-140; Prodoehl, J. A., Rhodes, A. L., Commings, R. S., Meller, M., H. H. Sherk: 308 nm Excimer laser ablation of cartilage, Lasers Surg. Med. 15 (1994) 263-268]. Ocular surgery applications include cataract surgery [Martinez, M., Maguen, E., Bardenstein, D., Duffy, M., Yoser, S., Papaioannou, T., W. Grundfest: A comparison of Excimer laser (308 nm) ablation of the human lens nucleus in air and saline with a fiber optic delivery system, Refract. Corneal Surg. 8 (1992) 368-374] and laser ablation of trabecular meshwork for glaucoma treatment [Vogel, M., K. Lauritzen: Selective Excimer laser ablation of the trabecular meshwork, Clinical results, Ophthalmologe 94 (1997) 665-667 and Walker. R, Specht. H:Theoretical and physical aspects of Excimer laser trabeculotomy (ELT) ab interno with the AIDA laser with a wavelength of 308 mm, Biomed Tech 2002 May;47(5):106-10].

The laser is preferably a pulsed laser. The pulse duration is not restricted and may be as short as 1 fs, for example in the range of 1 fs to 100 ps. The preferred pulse durations are within the range of 10 to 300 ns, more preferably 30 to 150 ns, most preferably 60 to 80 ns. For 308 nm the pulse energy at the tissue to be ablated is preferably in the order of 3 to 300 mJ/mm², more preferably 25 to 50 mJ/mm², most preferably 35 to 40 mJ/mm².

The laser radiation is transported to the application site by means of light guides, e.g. optical fibers such as quartz fibers, or hollow light guides. In a preferred embodiment, the light guide is a fiber optic delivery system integrated in a laser tip. The diameter of the optical fibers is preferably 50 to 2000 μm, more preferably 100 to 800 μm, most preferably about 200 to 600 μm. The distal exit area of the light guide is preferably in the range of 0.002 to 3 mm², preferably 0.008 to 0.3 mm², most preferably about 0.03 mm².

For treatment, the isolated sheet is preferably held with a holding means (such as forceps), holding frames or via vacuum suction arrangements which leave a large portion of the tissue untouched (rather than being laid against a surface). This is especially important in the case of epithelium cells which are easily irreversibly damaged by mechanical forces. The tissue therefore is preferably held suspended in a gas or a liquid, (e.g., body fluids, sterile fluids.) The light guide is then directed to the side of the tissue sheet to be treated. During treatment, the angle between the laser tip (i.e. the surface of the light guide from which the laser light exits) and the surface of the tissue sheet may be between 0 to 90°, and more preferably 30 to 60°. The distance between the tip and the tissue can be varied from 0 to 3000 μm, and is preferably 50 to 200 μm. The surgeon can hold this distance and angle manually under direct visual control or more preferably visual control may be aided by a microscope, magnification lenses and/or an endoscope. The surgeon can be aided by mechanical or magnetic guidance systems connected to the means holding the isolated sheet or by control systems measuring the distance (and/or angle) and providing the surgeon with at least one of visual, audible or tactile feedback. For example, the instrument can be mechanically connected to the device allowing specific movement in dimensions parallel to the sheet to be treated. The surgeon can also be assisted by robotic surgical tools.

The surgical instrument guiding the light guide may include of an optical fiber or bundle of optical fibers surrounded in a cannula of rigid material like (for example) stainless steel or passing through the working channel of an endoscope. The instrument may be shaped to facilitate the introduction into a patient's body to the site of treatment of the tissue sheet. In retinal surgery, this may comprise a straight instrument. The tip of the instrument where the laser light exits the light guide may be shaped to allow close approximation of the light guide to the tissue sheet while avoiding contact of the instrument with the tissue sheet and minimizing the damage in case of accidental contact. This can be achieved by a bevelled tip of the instrument and/or light guide, where the sum of the bevel of the tip and the angle at which the axis of the instrument is positioned relative to the tissue sheet defines the angle between the laser tip and the surface of the tissue sheet. To avoid situations resulting in counter-intuitive beam paths after the light exits the light-guide, the tip of the instrument can be equipped with at least one of a reflective surface and a lens. If a bare fiber is used with a bevelled tip, polished at an angle significantly different from 90° (for example), the instrument is preferably operated in a liquid with an index of refraction not much lower, about equal or even higher than that of the light guide (in glass and quartz fibers the index of refraction is typically between about 1.2 and 1.6 for the wavelengths transmitted, while pure water has an index of refraction of about 1.2 to 1.5, depending on the wavelength) and having a low absorption coefficient at the laser light wavelength. Suitable liquids include physiological saline. Also, a bundle of fibers of small diameter can be used. While the individual fibers have perpendicular exit surfaces, the fibers are arranged with some fibers protruding further than others, so that their exit surfaces approximate a bevelled tip. Such a bundle preferably is packed densely (hexagonal) but can be formed to the desired shape, with circular shapes being easy to produce and rectangular or linear shapes being particularly useful for precisely defined treatment of particular areas of the sheet.

The instrument surrounding the light guide (cannula or endoscope) is preferably shaped with rounded edges to allow closer approximation and to avoid damage in case of accidental contact with the tissue sheet to be treated. The light-guide exit surface can end to form an approximately smooth surface with the tip of the instrument, protrude (especially preferable in the case of an endoscope) or be withdrawn.

When ablating choroidal tissue, the laser operating parameters, such as the energy output, are selected such that the choroidal tissue is substantially ablated or, preferably, completely ablated with the Bruch's membrane remaining essentially intact.

In some embodiments of the invention, it may not be necessary to completely ablate all of the choroidal tissue. For example, it may be possible to ablate or emulsify only the choroidal tissue surrounding vessels since the vessels, particularly the arteries, are less easily ablated/emulsified and are relatively strongly attached. Accordingly, after the choroidal tissue surrounding the vessels has been ablated/emulsified, the vessels may be easily removed by simply pulling the vessels from the sheet.

At any rate, the laser ablation treatment is discontinued before the RPE layer is ablated. To ensure this, the laser ablation is preferably controlled by monitoring one or more optical properties or one or more mechanical properties of at least one of the sheet of tissue and the ablation plume. When the monitored property changes significantly, the laser ablation is preferably stopped. Alternatively, the energy level of the next laser pulse may be adjusted accordingly. An audible, visual or tactile signal may be provided to inform the surgeon of this change. For example, at least one of the fluorescence and the reflection of the sheet of tissue can be monitored, where the fluorescence and reflection being induced by the laser used for ablation (including a pilot beam or pulse thereof) or by a second laser. The monitored mechanical property can be the sound or pressure waves emitted by the ablation.

During laser ablation, an ultraviolet absorbing compound may be administered to the patient to protect the eye from scattered laser light. Moreover, a compound that is useful for the therapeutic treatment of macular generation may also be administered. At least one of the ultraviolet absorbing and therapeutically active compound is preferably administered intraocularly. These compounds include glutathione enhancing agents as disclosed in U.S. Pat. No. 5,596,011, such as N-acetyl cysteine; vitamins such as ascorbic acid, alpha-tocopherol (vitamin E), beta-carotone, retinal (vitamin A), lutein, zeaxanthin; and inhibitors of the protein tyrosine kinase pathway such as genistein.

After the laser ablation, the treated sheet is inserted to the location from which the sheet of tissue was excised or to another location. It is particularly preferred to translocate the treated sheet to another location from which another tissue has been excised (e.g. damaged or degenerated tissue). In such a case, the surgical method of the present invention is a transplantation method, i.e. the sheet of tissue is translocated or transplanted to the location from which the damaged or degenerated tissue (e.g. the choroidal neovascularizations or neovascular membranes) was excised. In the case of treating choroidal neovascularizations, e.g. those associated with age-related macular degeneration, this is preferably done in a manner such that the Bruch's membrane of the treated sheet of tissue is in contact with the Bruch's membrane of the location from which the choroidal neovascularizations were excised. Hence, the present invention allows the translocation of intact autologous RPE sheets.

After these steps, the surgery may be completed in ways evident to vitreoretinal surgeons and others skilled in the art of ophthalmic surgery. In that regard, completion of the surgery may include using a temporary internal tamponade which is either silicon oil or air and C3F8 gas (e.g., C3F8 or SF6). These tamponades are commonly used in vitreoretinal surgery.

In the first step of the transplantation method of the present invention, damaged or degenerated tissue is excised. The damaged or degenerated tissue preferably comprises (subfoveal) choroidal neovascularization (CNV). It is preferably excised from the neurosensory retina, and most preferably from the macular region (macula lutea), that may be associated with late-stage age-related macular degeneration.

The choroidal neovascularizations (CNV) may be identified and subsequently removed as follows: Sequalae of choroidal neovascularizations such as hemorrhage within and/or under the retina as well as swelling of the retina (macular edema) can be seen by funduscopy. The neovascular membranes are identified with the help of fluorescein and indocyanine green angiography. The neovascular net is then removed by performing a small retinotomy temporal to the macula. Subsequently, balanced salt solution may be injected into the subretinal space to separate the neurosensory retina from the RPE and the neovascular membrane at the posterior pole. Subretinal forceps may then be introduced into the subretinal space and the CNV is removed via the retinotomy site and, subsequently, through a sclerotomy. The surgical excision of CNV is usually associated with the removal of corresponding retinal pigment epithelium (RPE), so that photoreceptors will be in direct contact with Bruch's membrane. This will inevitably lead to irreversible degeneration and cell death of the neuronal outer retinal cellular elements.

Thus, in a most preferred embodiment, the present invention thus provides a method including excising choroidal neovascularizations (neovascular membranes) from the neurosensory retina, isolating a sheet of tissue comprising choroidal tissue, Bruch's membrane and retinal pigment epithelial cells from the peripheral retina, treating the isolated sheet of tissue with a laser light thereby ablating the choroidal tissue and transplanting the treated sheet of tissue (which may include the retinal pigment epithelial cells) to the location from which the choroidal neovascularizations (neovascular membranes) were excised.

This method is particularly useful for the treatment of one of more of the following conditions: macular degeneration, histoplasmosis, pathological myopia, angioid streaks, anterior ischemic optic neuropathy, bacterial endocarditis, Best's disease, birdshot retinochoroidopathy, choroidal hemangioma, choroidal nevi, choroidal nonperfusion, choroidal osteomas, choroidal rupture, choroideremia, chronic retinal detachment, coloboma or the retina, Drusen, endogenous Candida endophthalmitis, extra-papillary hamartomas of the retinal pigmented epithelium, fundus flavimaculatus, idiopathic (sic), macular hold, malignant melanoma, membrane proliferative glomerulonephritis (type II), metallic intraocular foreign body, morning glory disc syndrome, multiple evanescent white-dot syndrome, neovascularization at ora serrata, operating microscope burn, optic nerve head pits, photocoagulation, punctate inner choroidopathy, radiation retinopathy, retinal cryoinjury, retinitis pigmentosa, retinochoroidal coloboma, rubella, sarcoidosis, serpoiginous or geography choroiditis, subretinal fluid drainage, titled disc syndrome, Taxoplasma retinochoroiditis, tuberculosis and Vogt-Koyanagi-Harada syndrome.

### EXAMPLE

Method:

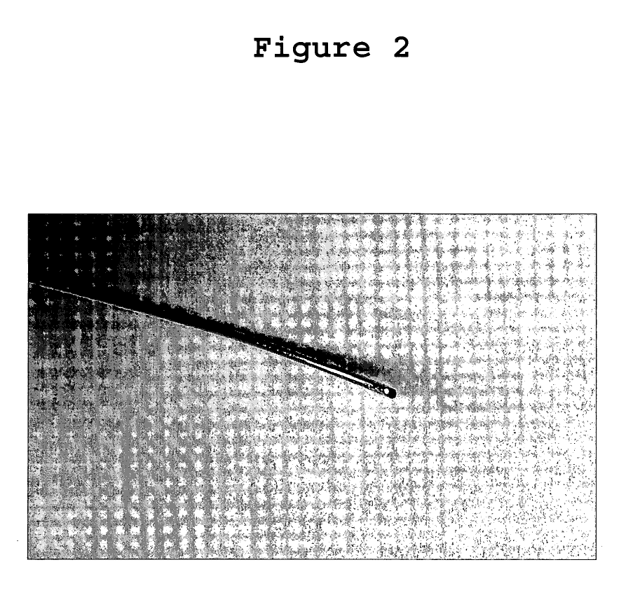

Freshly enucleated human donor eyes that were obtained for corneal transplantation were used. The anterior portion including the cornea, the lens, the iris and anterior sclera was excised and the remaining open eye cup placed on an eye cup holder under a photomicroscope (Wild, Herbrugg, Switzerland). The eye was filled with balanced saline solution (BSS) and illuminated by a flexible halogen fiber optic (Schott, Germany). A fiber optic delivery system integrated in a laser tip and a 308 nm AIDA UV-Excimer laser (Glautec AG, Switzerland) were used. The laser tip showed an angle of approx. 45° (FIG. 2). The rectangular grinded laser tip aperture was 7×30 μm². Laser energy was modified using an infinitely variable aperture. Energy ranged from 0.19 to 0.38 μJ. Pulse length was 60 ns. The retina was removed by small forceps and a 3×3 mm² graft of RPE, Bruch's membrane and choroid was excised. The graft was held with small forceps and the laser tip forwarded towards the choroidal side in a distance between 1 to 3 mm. Photographs of ablated choroid were taken using the photomicroscope and a camera (Olympus BX 50, Hamburg, Germany). Ablated RPE/choroid tissue was fixed in formaldehyde, dehydrated by ethanol, stained with hematoxilin-eosin and embedded in paraffin for light microscopy. For electron microscopy the tissue was fixed for 2 hours with 3% glutaraldehyde in 0.1 M cacodylate buffer, post fixed for 2 hours in 1% osmium tetroxide in 0.1 M cacodylate buffer before being dehydrated in ethanol and propylenoxide. Afterwards the tissue was embedded in Epon and Araldit (Serva) for 3 days. Sections were cut on a Reichert-Jung ultramicrotome and mounted on copper grids (Plano, Wetzlar, Germany) before staining with uranyl and lead citrate. Electron microscopy was performed using an Elo-TSD, Zeiss, Germany).

Results:

During the laser treatment, laser pulses at the tip created a dim blue flash and, depending on the energy applied, small gas bubbles were noted. A broad spectrum of laser energy (0.5 mJ to 1.0 mJ for the 200 μm laser fibre, 1.2 to 2.1 mJ for the 600 μm laser fibre) was used to ablate effectively due to large interindividual differences in tissue diameters and properties. Accidental contacts of the laser tip did not damage the layer and caused no perforation. Treated specimen were cut by a scalpel and fixed as described in the methods section. Light microscopic examination showed cleanly and accurately separated tissue borders without thermal collateral damage. Measurements of choroidal thickness showed a reduction of up to 80% compared with untreated specimen from the same eye. Variable amounts of inner choroidal tissue was still present at the basal side of Bruch's membrane. Morphologically the overlying RPE cells appeared normal. Electron microscopy confirmed the result of light microscopy.

Discussion:

In this experimental study we demonstrated that choroidal tissue can be effectively microablated from a RPE/Bruch's membrane/choroid graft with a 308 nm UV-Excimer laser. A probe was designed and used in these experiments that could also be applied during surgery for intraocular ablation. Application of this technique for RPE-transplantation in eyes with neovascular age-related macular degeneration may yield better functional results when compared with grafts that have normal, unablated choroidal tissue. It has been demonstrated that it is possible to precisely ablate tissue without thermal or other collateral damage. This may relate to the small ratio between radiation-absorbing tissue to water. Non-contact laser-guided removal of tissue allows the application of laser effects without mechanical damage to the tissue.

Recently autologous RPE/Bruch's membrane choroid grafts have been translocated in eyes with prior excision of subfoveal neovascular membranes that led to markedly impaired central visual function before intervention. Hereby, the goal is to transplant functional RPE-cell monolayer sheets with minimal trauma under the neurosensory retina. Minimization of trauma and maintenance of an intact RPE monolayer appears crucial as RPE cells may otherwise dedifferentiate, and, consequently, loose their functional properties essential for normal photoreceptor function. Both subretinal injection of autologous RPE or iris pigment epithelial cells did no form results as satisfactory as desired, as these cell suspensions were lacking adequate formation of monolayers, but showed clumping and apparent dedifferentiation [Binder, S., Stolba, U., Krebs, I.,Kellner, L., Jahn, C., Feichtinger, H., Povelka, M., Frohner, U., Kruger, A., Hilgers, R. D., W. Krugluger: Transplantation of autologous retinal pigment epithelium in eyes with foveal neovascularization resulting from age-related macular degeneration: A pilot study, Am. J. Ophthalmol. 133 (2002) 215-225]. Therefore, the use of grafts with supporting extracellular matrix such as Bruch's membrane has a good rational. However, the choroid is firmly attached to Bruch's membrane or the overlying RPE cell layer. The use of the 308 nm Excimer laser allows the intraocular treatment without bringing the graft out of the intraocular milieu in an adverse environment that might traumatize the RPE cells and cause later dysfunction when repositioned under the retina. The laser probe here may be introduced into the vitreous cavity via the small openings of normal 0.9 mm sclerotomies that are created regularly for vitreoretinal surgery. Thus, choroidal tissue from the graft could be ablated within the eye.

The AIDA UV-Excimer laser thus is a suitable tool for an atraumatic thinning of the choroid. The interaction between laser light and tissue depends on absorption coefficient of target tissue, time constants (e.g. exposure time) and delivered energy. The wavelength of 308 nm has an absorption coefficient of 0.01 cm⁻¹ in water. This equals a penetration of 1 meter. The absorption coefficient of tissue is much more larger (50 cm⁻¹).

The resulting optical penetration is less than 200 μm. These small ablation rates in the range of μm facilitate an atraumatic preparation of RPE/choroid complexes due to controlled removal of tissue. Furthermore, the AIDA UV-Excimer laser shows only 2 μm diffusion of thermal energy per pulse. Thermal damage is therefore limited to the boundaries of laser treatment, leaving overlying RPE intact. Gas bubble generation is not caused by explosive water evaporation but related to slow heat transport from tissue to water.

In summary, the method of the present invention is a promising way to ablate intraocularly prepared RPE/choroid transplants that might improve visual outcome of patients with neovascular age-related macular degeneration.

What is claimed is:

1. A surgical method, comprising:
excising a first sheet of tissue from surrounding tissue at a particular location;
treating the excised sheet of tissue with focused energy to ablate at least one layer of the sheet, or a part thereof; and
inserting the treated sheet back to the location or to another location.

2. The surgical method according to claim 1, wherein the focused energy comprises at least one of laser light, ultrasound and high-frequency electrical energy.

3. The surgical method according to claim 1, wherein the focused energy comprises laser light supplied by a laser operated at a wavelength in the range of about 180 to about 400 nm.

4. The surgical method according to claim 3, wherein the wavelength is either about 193 nm or about 308 nm.

5. The surgical method according to claim 3, wherein the laser is an Excimer laser.

6. The surgical method according to claim 3, wherein the laser is either a 193 nm or a 308 nm Excimer laser.

7. The surgical method according to claim 3, wherein the laser light is delivered in pulses with pulse duration of between about 10 ns to about 300 ns.

8. The surgical method according to claim 7, wherein the pulse energy is in the range of about 3 to about 300 mJ/mm².

9. The surgical method according to claim 3, wherein the laser light is applied via a light guide.

10. The surgical method according to claim 9, wherein the light guide is a quartz fiber or a hollow light guide.

11. The surgical method according to claim 9, wherein the light guide is a fiber optic delivery system integrated in a laser tip.

12. The surgical method according to claim 11, wherein the angle between the laser tip and the surface of the sheet of tissue is about 30 to about 60°.

13. The surgical method according to claim 9, wherein the distal exit area of the light guide is in the order of about 0.002 mm² to about 3 mm².

14. The surgical method according to claim 1, wherein the focused energy ablates and/or emulsifies tissue intraocularly.

15. The surgical method according to claim 1, wherein the surface area of one side of the isolated sheet is about 1 mm² to about 30 mm².

16. The surgical method according to claim 3, further comprising controlling the ablation by monitoring one or more optical properties of the sheet of tissue, or of an ablation plume, and wherein the focused energy supplied by the laser is substantially halted or wherein the energy level of the next pulse is adjusted accordingly when the one or more optical properties change significantly,

17. The surgical method according to claim 16, wherein the optical property includes at least one of the fluorescence and the reflection of at least one of the sheet of tissue and the ablation plume, and wherein at least one of the fluorescence and the reflection being induced by the laser, via a pilot beam or pulse thereof, or by a second laser.

18. The surgical method according to claim 16, wherein at least one of an audible, visual and tactile signal is provided informing the surgeon that one or more optical properties have changed significantly.

19. The surgical method according to claim 3, wherein the method further comprises controlling the ablation by monitoring one or more mechanical properties of the sheet of tissue or of the ablation plume, and wherein the laser is substantially halted or the energy level of the next pulse is adjusted accordingly when the one or more mechanical properties change significantly.

20. The surgical method according to claim 19, wherein the mechanical properties are monitored by analysing at least one of the sound or the pressure waves emitted by the ablation.

21. The surgical method according to claim 19, wherein at least one of an audible, visual or tactile signal is provided informing the surgeon that one or more mechanical properties have changed significantly.

22. A surgical method comprising:
excising damaged and/or degenerative tissue from a particular location;
isolating from the damaged and/or degenerative tissue a sheet of non-damaged and/or non-degenerated tissue comprising at least two layers of tissue;
treating the isolated sheet with focused energy to ablate at least one layer of tissue or a part thereof; and
inserting the treated sheet of tissue back to the location.

23. The surgical method according to claim 22, wherein the focused energy comprises at least one of laser light, ultrasound and high-frequency electrical energy.

24. A surgical method comprising:
excising tissue comprising at least one choroidal neovascularization from a particular location;
isolating a sheet of tissue comprising choroidal tissue from the excised tissue;
treating the isolated sheet of tissue with focused energy to ablate all or a part of the choroidal tissue;
inserting the treated sheet of tissue back to the location.

25. The surgical method according to claim 24, wherein an ultraviolet absorbing compound is administered to the sheet during the laser ablation.

26. The surgical method according to claim 24, wherein the focused energy comprises at least one of laser light, ultrasound and high-frequency electrical energy.

27. A surgical method comprising:
excising at least one choroidal neovascularization from a neurosensory retina at a particular location;
isolating a sheet of tissue comprising choroidal tissue, Bruch's membrane and retinal pigment epithelial cells;
treating the isolated sheet with focused energy to ablate all or a part of the choroidal tissue; and
inserting the treated sheet which includes the retinal pigment epithelial cells back to the location.

28. The surgical method according to claim 27, wherein the sheet of tissue including the retinal pigment epithelial cells and the essentially intact Bruch's membrane is transplanted to the location from which the choroidal neovascularization was excised such that the Bruch's membrane of the sheet of tissue is in contact with the Bruch's membrane of the location from which the choroidal neovascularization was excised.

29. The surgical method according to claim 27, wherein the focused energy is laser light supplied by a laser, and wherein the energy of the laser is selected such that a substantial portion of the Bruch's membrane remains essentially intact.

30. The surgical method according to claim 27, wherein the focused energy comprises at least one of laser light, ultrasound and high-frequency electrical energy.

31. A method for the treatment of macular degeneration comprising:
excising at least one choroidal neovascularization from a particular location;
isolating a sheet of tissue from the choroidal neovascularization comprising choroidal tissue, Bruch's membrane and retinal pigment epithelial cells from the peripheral retina;
treating the isolated sheet of tissue with an Excimer laser thereby ablating all or a part of the choroidal tissue; and
inserting the treated sheet of tissue including the retinal pigment epithelial cells and the Bruch's membrane back to the location.

32. The surgical method according to claim 31, wherein the choroidal neovascularization is excised from the Macula lutea.
